(19) United States
(12) Patent Application Publication
(10) Pub. No.: US 2005/0216025 A1
(43) Pub. Date: Sep. 29, 2005

(54) Device for forming a hardened cement in a bone cavity

(75) Inventors: Jiin-Huey Chern Lin, Winnetka, IL (US); Chien-Ping Ju, Carbondale, IL (US)

Correspondence Address: BACON & THOMAS, PLLC, 625 SLATERS LANE, FOURTH FLOOR, ALEXANDRIA, VA, 22314, US

(73) Assignee: CANA LAB CORPORATION, TAIPEI (TW)

(21) Appl. No.: 10/805,255

(22) Filed: Mar. 22, 2004

(51) Int. Cl. A61B 17/58

(52) U.S. Cl. 606/92

(57) ABSTRACT

A device for injecting a cement paste into a bone cavity and forming a hardened cement in-situ under pressure includes a syringe having an injection end; a pocket having an inlet and a body for containing the paste entering the inlet; and a mounting mechanism at the injection end for connecting the inlet of the pocket to the injection end so that the body of the pocket can contain the paste through the syringe. The device further includes a leaking mechanism provided at the injection end of the syringe for allowing liquid contained in the paste inside the pocket to be expelled from the pocket under pressure, and an opening mechanism which can be operated at a location away from the injection end to open the body of the pocket.

### FIELD OF THE INVENTION

The present invention is related to a technique of forming a set or hardened bone cement in bone cavity, and in particular to a technique of hardening a bone cement under an exerted pressure in a bone cavity.

### BACKGROUND OF THE INVENTION

Calcium phosphate cement (abbreviated as CPC) has been widely used as an implant or filling material in dental and bone prosthesis, and its technical details can be found in many patents, for examples U.S. Pat. Nos. 4,959,104; 5,092,888; 5,180,426; 5,262,166; 5,336,264; 5,525,148; 5,053,212; 5,149,368; 5,342,441; 5,503,164; 5,542,973; 5,545,254; 5,695,729 and 5,814,681. Similar to CPC, calcium sulfate and bioactive glass have also been suggested or used as an implant or filling material in dental and bone prosthesis.

Heretofore the conventional method of forming a set or hardened bone cement in bone cavity involves directly injecting a cement paste into bone cavity, which suffers the followings drawbacks among others:
(1) While the liquid-powder ratio of the cement paste is too high, the strength of the hardened cement becomes too low, that can cause the cement to more easily disperse/disintegrate;
(2) While the liquid-powder ratio of the cement paste is too low, the viscosity of the paste becomes too high, the working and setting times become too short, and the paste is hard to inject through a syringe;
(3) Dispersed cement particles in body fluid/blood, especially before being fully set, can penetrate into surrounding tissue, that can cause serious hazard during or after surgery.

### SUMMARY OF THE INVENTION

A primary objective of the present invention is to provide a method and a device for forming a hardened cement in a bone cavity, which are free of the aforesaid prior art drawbacks.

The present invention provides a device for forming a hardened cement in a bone cavity comprising a syringe having an injection end; a pocket having an inlet and a body for containing a paste entering said inlet; and a mounting mechanism at said injection end for connecting said inlet of said pocket to said injection end so that said body of said pocket can contain said paste through said syringe; characterized in that the device further comprises a leaking mechanism provided at said injection end of said syringe for allowing liquid contained in the paste inside said pocket to be expelled from said pocket under pressure, and an opening mechanism which can be operated at a location away from said injection end to open said body of said pocket.

Preferably, said syringe comprises a tube and an injector plug movably received in said tube, wherein said tube is provided with one or more longitudinal grooves on an outside surface thereof at an injection end thereof; and said pocket comprises a neck defining said inlet, wherein said injection end of said tube is inserted into the inlet of said pocket and said neck of said pocket is fastened to the outside surface of the tube by said mounting mechanism, so that said one or more longitudinal grooves form one or more gaps between the neck of said pocket and the outside surface of said tube of said syringe, and said leaking mechanism comprises said one or more gaps. Alternatively, said one or more longitudinal grooves are provided on said injector plug or on an inside surface of said tube at an injection end thereof, so that said one or more longitudinal grooves form one or more gaps between the injector plug and the inside surface of said tube of said syringe, when the injector plug reaches said injection end of said tube, and said leaking mechanism comprises said one or more gaps. Preferably, the device of the present invention further comprises a hole provided on said tube, said hole being adapted to be connected to a vacuum facility, so that said liquid expelled from said pocket can be suck out via said hole when said vacuum facility is driven.

Preferably, said pocket is made of an elastic polymeric material, so that the body of said pocket can be dilated with the paste inside said pocket under pressure; and said opening mechanism comprises an electrically conductive wire of high electric resistance, said wire having two ends being adapted to connect to a positive electrode and a negative electrode of a power supply, respectively, and one or more points between said two ends of said wire being attached to the body of said pocket, so that the dilated pocket body will rupture due to melting or weakening of the elastic polymeric material caused by a heat generated at the attached wire, when the ends of said wire are connected to the positive electrode and the negative electrode of the power supply.

Preferably, said pocket is made of an elastic polymeric material, and said opening mechanism comprises a first set of wire holders on an outer surface of said syringe, which are spaced apart along a longitudinal direction of said syringe; a second set of wire holders on said outer surface of said syringe, which are spaced apart along said longitudinal direction of said syringe, wherein an imaginary plane formed by said first set of wire holders and said second set of wire holders divides the syringe into halves; and said thin wire which is slidably received said first set of wire holders and said second set of wire holders with a portion thereof passing across said injection end of said syringe, thereby said pocket can be cut to rupture with a sliding movement of said thin wire.

Preferably, said pocket is made of an elastic polymeric material, and said opening mechanism comprises a thin tube on an outer surface of said syringe along a longitudinal direction of said syringe; and a knife slidably received in said thin tube, said knife having a retractable blade and a rod connected to said retractable blade at one end thereof, so that said retractable blade received in said tube is able to protrude from said injection end of said syringe by pushing the rod, and thus said pocket can be cut to rupture by said retractable blade, and that said protruding retractable blade can be retracted by pulling the rod.

The present invention solves the aforesaid prior art drawbacks, because the cement paste set within the closed pocket without contacting directly body fluid/blood, and pressure can be applied/developed within the pocket, which will increase largely the strength of the cement, reduces the risk of cement dispersion/disintegration, and also avoid “cement paste leaking”.

Further, the present invention has an advantage of being easy to keep a powder/liquid ratio of the cement paste accurate by monitoring the pressure build-up within the pocket, that is important to cement properties such as setting time and strength.

### DETAILED DESCRIPTION OF THE PREFERRED EMBODIMENTS

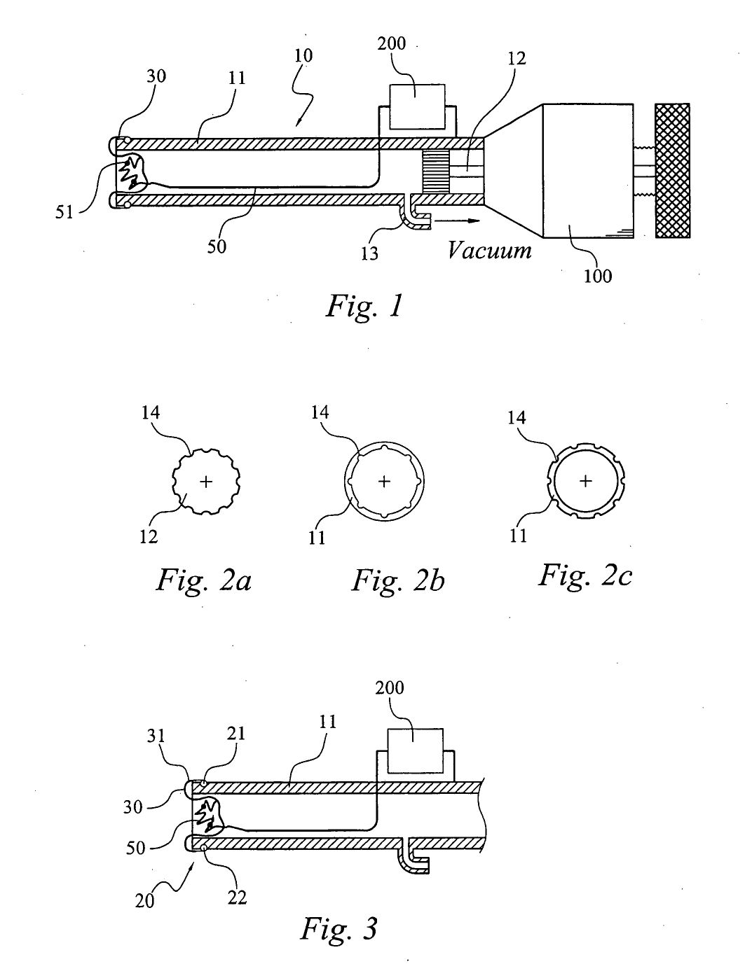

FIG. 1, FIGS. 3 to 6 are cross-sectional views of a device for forming a hardened cement in a bone cavity constructed according to a first preferred embodiment of the present invention, and together show a process flow diagram of the method of the present invention.

FIG. 2a is a lateral cross-sectional view of the plug 12 depicted in FIG. 1.

FIG. 2b is a lateral cross-sectional view of a tube 11 suitable for use in the device of the present invention, wherein grooves 14 are provided on an inside surface thereof.

FIG. 2c is a lateral cross-sectional view of a tube 11 suitable for use in the device of the present invention, wherein grooves 14 are provided on an outside surface thereof.

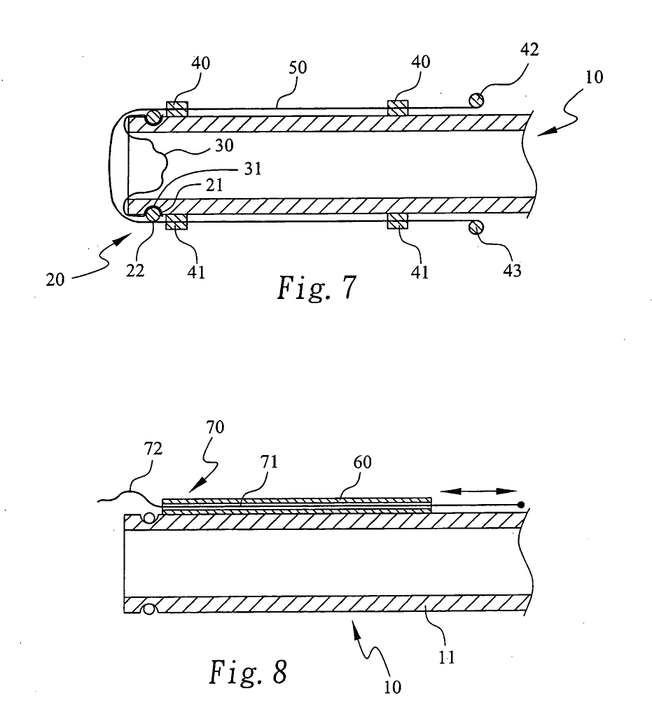

FIGS. 7 is a cross-sectional view of a device for forming a hardened cement in a bone cavity constructed according to a second preferred embodiment of the present invention, and together show a process flow diagram of the method of the present invention.

FIG. 8 is a cross-sectional view of a device for forming a hardened cement in a bone cavity constructed according to a third preferred embodiment of the present invention.

### DETAILED DESCRIPTION OF THE PREFERRED EMBODIMENTS

A device for forming a hardened cement in a bone cavity constructed according to a first preferred embodiment of the present invention is shown in FIGS. 1 to 6, which will elaborated as follows:

A. System Description

The bone cement delivery tool described below consists of the following major components, as shown in FIG. 1:
1) A reservoir 100 which can contain the cement paste to be delivered into the bone cavity;
2) An injector plug 12 which can be used to push the cement paste through a slender cylindrical tube 11;
3) The cylindrical tube 11 which serves as the conduit for cement injection. The tube 11 and the injector plug 12 form a syringe 10. The tube can be drilled with a side hole 13 to access to a vacuum facility;
4) A rubber-type balloon 30 attached to the proximal end (injection end) of the tube 11. This balloon 30 can hold the cement paste against the wall of the bone cavity;
5) An electric wire 50 of high electric resistance. The two points 51 at the middle section of this electric wire is embedded in the balloon 50 whereas the two ends are connected to a power supply 200 having a switch.

The rubber-type balloon 30 should have appropriate elastic property which allows the balloon expand 3-5 times during volume dilation. As cement paste enters inside the balloon 30, the balloon can hold the cement non-permeably while fitting tightly against the bone cavity wall. The balloon thickness should be selected with sufficient strength when expanded to its intended dilated volume. This dilated balloon should develop appropriate tension to facilitate its rupture and shrinkage as described below. However, the developed tension should not be too large so as to avoid premature balloon rupture. The balloon 30 can be manufactured using those techniques available in the design of intra-vascular balloon catheter devices, for instance, the technique of solution casting of polyurethane or other polymers.

On the outer surface of the balloon 30, there is the naked high-resistance electric wire 50 attached. The length of between the points 51 of the wire 50 should be calculated using the maximally dilated balloon configuration. More than two attachment points can be used to fix the wire 50 onto the balloon 30. The balloon 30 can be formed by casting and curing a liquid polyurethane on a mold. Two or more than two points of the electric wire 50 can be embedded in the polyurethane before the solidification of the polyurethane, or glued to the surface of the balloon by an adhesive after the solidification of the polyurethane.

As the power supply 200 is turned on, the temperature of the electric wire 50 will be developed and heat released to melt the balloon material in contact with the electric wire 50. The dilated balloon 30 will soon be ruptured and shrunk back to its original zero-stress state. This rupturing constitutes automatically an extraction function of the balloon 30. The higher the tension in the balloon, the more effective the rupture and back extraction of the balloon. However, care must be exercised to avoid excessive tension developed which may promote undesired premature balloon rupture caused by contacting with the rough surface of the bone cavity during its cement delivery period.

The pressurization of the delivered cement has two major functions that characterize the present method. The first is the function of expanding the collapsed bone structure to some desired shape and size. Through the fluid motion of the cement paste, pressure can be transmitted to make the cement fill tightly within the bone cavity and in the same time push the bone structure restoring back to its original shape and size. Secondly, during the pressurization of the cement, water content of the cement can be squeezed out of the balloon 30 via a groove system made on the wall of the injector plug 12 or on the cylindrical tube 11 as shown in FIGS. 2a, 2b and 2c, wherein longitudinal grooves 14 are formed on the injector rod 12, inside surface of the cylindrical tube 11, and outside surface of the cylindrical tube 11, respectively. For the design in FIGS. 2a and 2b, water can be forced out using the tube as a drainage duct, wherein a vacuum facility can be connected to the hole 13 to suck out the water. For the design depicted in FIG. 2c, however, water can be drained along the grooves 14 carved on the outer surface of the tube 11 and absorbed by the human body. Pressurization and water extraction will help the solidification of the cement, which is critical for the structure and strength development as the cement is dried within the balloon 30.

B. Delivery and Formation of Cement

1) Before delivering an appropriate amount of cement paste into a bone cavity using the long slender cylindrical tube 11 and an injector plug 12, the balloon 30 with the attached electric wire 50 is connected to the injection end of the cylindrical tube 11 by a mounting mechanism 20 having an annular groove 21 provided on an outer surface of the tube 11, and a ring 22 adapted to elastically grip the annular groove 21. The injection end of the cylindrical tube 11 is inserted into an opening of the balloon 30, so that a neck 31 of the balloon 30 covers the annular groove 21; and putting the ring 22, which is a closed ring or a C-shaped ring, on the neck 31 of the balloon 30 and clamping it at the annular groove 21 on the cylindrical tube 11, as shown in FIG. 3;

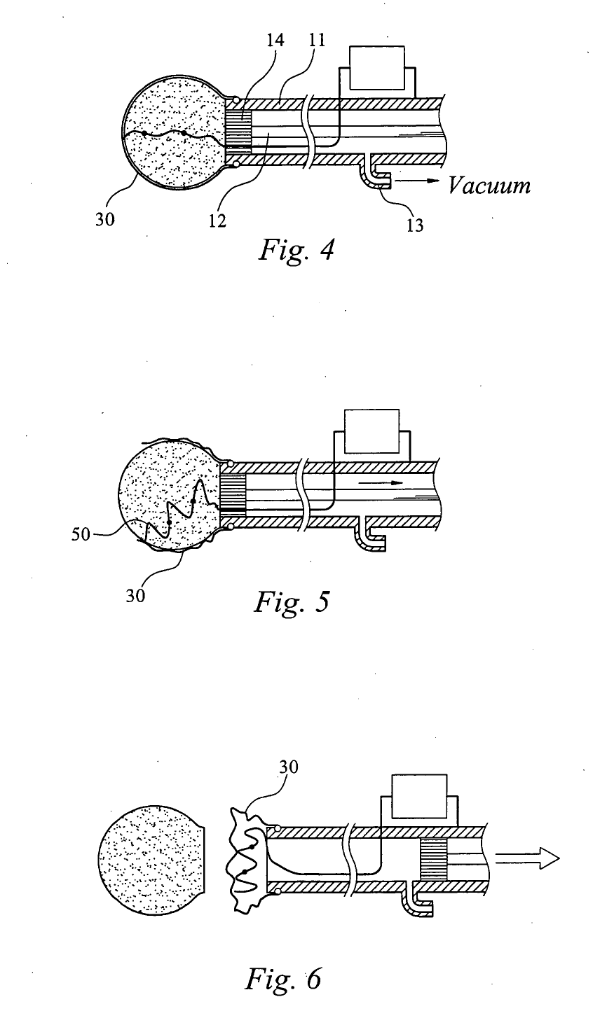

2) Expand the balloon 30 by pushing the injector plug 12 to compress the cement paste with a pre-calibrated pressure until the damaged bone is expanded to the desired shape and size as shown in FIG. 4, wherein liquid expelled from the cement paste via the grooves 14 is suck out using the tube 11 as a drainage duct by connecting a vacuum facility to the hole 13;

3) Pressurize the cement paste for a period of time until the cement is dried and hardened in the balloon 30;

4) Electrify the high-resistance electric wire 50 to rupture the balloon 30, as shown in FIG. 5;

5) Extract the ruptured balloon 30 while holding the injector plug against the cavity outlet until the balloon 30 clears the exit; and

6) Withdraw the whole delivery system out of the patient body as shown in FIG. 6.

A device for forming a hardened cement in a bone cavity constructed according to a second preferred embodiment of the present invention is shown in FIG. 7, in which parts having a similar function to parts shown in FIG. 1 have been given similar reference numerals. The device contains a syringe 10 having a substantially cylindrical tube 11 and a plug identical to the one used in the first embodiment (not shown in FIG. 8) slidably received in the tube 11; and a mounting mechanism 20 having an annular groove 21 provided on an outer surface of the tube 11, and a ring 22 adapted to elastically grip the annular groove 21. A balloon 30 is mounted to the injection end of the syringe with the ring 22.

The device further contains a first set of wire holders 40 on the outer surface of the cylindrical tube 11 and along the longitudinal direction thereof; and a second set of wire holders 41 on the outer surface of the cylindrical tube 11, which are symmetrical to the first set of wire holders 40; and a thin wire 50′ slidably received in the first and second sets of wire holders 40 and 41. The wire holders 40 and 41 are blocks each having a through hole, through which the thin wire 50′ is passed and guided longitudinally. Preferably, the device further has two tension-adjustable rollers 42 and 43, on which the ends of the thin wire 50 are wound, so that the thin wire 50′ is maintained in the first set and second set of wire holders 40 and 41 under a controlled tension. The device of the present invention is now ready to be used. The injection end of the syringe 10 is inserted into a bone cavity through an incision cut and a hole drilled by the operator. A cement paste, preferably a CPC paste giving a setting time less than 20 minutes, more preferably less than 10 minutes, is injected into the balloon 30 by pushing the plug in the tube 11 toward the injection end of the syringe, so that the balloon 30 is inflated and the portion of the thin wire 50′ passing across the injection end is pushed, and thus the thin wire 50′ is un-wound from one or both of the rollers 42 and 43 until all the CPC paste is injected into the balloon 30. The CPC paste in the balloon 30 is maintained under the pressure exerted by the plug while setting with a reduced liquid/solid ratio due to leakage of liquid via meshes of the fiber cloth of the balloon 30, and preferably the pressure is about 1-5000 psi, and more preferably 10-1000 psi. The thin wire 50′ is pulled forward and backward alternatively at its ends under tension to cut the balloon 30 after the CPC paste is hardened in the balloon 30. One end of the thin wire 50′ is released from the roller 42 by continuously pulling the thin wire 50′ with the roller 43, after the balloon 30 is cut open. Finally the hardened CPC is left in the bone cavity by retreating the device together with the opened balloon 30 from the patient.

The cutting of the balloon 30 can be carried out by a different cutting structure. FIG. 8 shows a modified device of the present invention based on the design shown in FIG. 7A, wherein like elements or parts are represented by like numerals. A thin tube 60 is provided on the outer surface and along a longitudinal direction of the cylindrical tube 11 of the syringe 10. A knife 70 having a rod 71 and a retractable blade 72 is slidably received in the thin tube 60 by inserting the rod 71 into the thin tube 60 from the end near the injection end of the syringe 10 until the retractable blade 72 enters the thin tube 60. The retractable blade 72 is preferably made of metal and is elastic, so that it resumes its shape after being pushed to protrude from the thin tube 60. The operator can grip the rod 71 from the other end of the thin tube 60 to push the retractable blade 72 to protrude from the thin tube 60, cut the balloon 30 with the retractable blade 72, and retract it once again.

It is apparent that the cutting structure shown in FIG. 8 can be incorporated to the device shown in FIG. 7 to assure a successful cutting of the balloon.

Although the present invention has been described with reference to specific details of certain embodiments thereof, it is not intended that such details should be regarded as limitations upon the scope of the invention except as and to the extent that they are included in the accompanying claims. Many modifications and variations are possible in light of the above disclosure.

1. A device for forming a hardened cement in a bone cavity comprising a syringe having an injection end; a pocket having an inlet and a body for containing a paste entering said inlet; and a mounting mechanism at said injection end for connecting said inlet of said pocket to said injection end so that said body of said pocket can contain said paste through said syringe; characterized in that the device further comprises a leaking mechanism provided on said body of said pocket or provided at said injection end of said syringe for allowing liquid contained in the paste inside said pocket to be expelled from said pocket under pressure, and an opening mechanism which can be operated at a location away from said injection end to open said body of said pocket.

2. The device according to claim 1, wherein said syringe comprises a tube and an injector plug movably received in said tube, wherein said tube is provided with one or more longitudinal grooves on an outside surface thereof at an injection end thereof; and said pocket comprises a neck defining said inlet, wherein said injection end of said tube is inserted into the inlet of said pocket and said neck of said pocket is fastened to the outside surface of the tube by said mounting mechanism, so that said one or more longitudinal grooves form one or more gaps between the neck of said pocket and the outside surface of said tube of said syringe, and said leaking mechanism comprises said one or more gaps.

3. The device according to claim 1, wherein said syringe comprises a tube and an injector plug movably received in said tube, wherein one or more longitudinal grooves are provided on said injector plug or on an inside surface of said tube at an injection end thereof; and said pocket comprises a neck defining said inlet, wherein said injection end of said tube is inserted into the inlet of said pocket and said neck of said pocket is fastened to the outside surface of the tube by said mounting mechanism, so that said one or more longitudinal grooves form one or more gaps between the injector plug and the inside surface of said tube of said syringe, when the injector plug reaches said injection end of said tube, and said leaking mechanism comprises said one or more gaps.

4. The device according to claim 3 further comprising a hole provided on said tube, said hole being adapted to be connected to a vacuum facility, so that said liquid expelled from said pocket can be suck out via said hole when said vacuum facility is driven.

5. The device according to claim 1, wherein said pocket is made of an elastic polymeric material, so that the body of said pocket can be dilated with the paste inside said pocket under pressure; and said opening mechanism comprises an electrically conductive wire of high electric resistance, said wire having two ends being adapted to connect to a positive electrode and a negative electrode of a power supply, respectively, and one or more points between said two ends of said wire being attached to the body of said pocket, so that the dilated pocket body will rupture due to melting or weakening of the elastic polymeric material caused by a heat generated at the attached wire, when the ends of said wire are connected to the positive electrode and the negative electrode of the power supply.

6. The device according to claim 2, wherein said pocket is made of an elastic polymeric material, so that the body of said pocket can be dilated with the paste inside said pocket under pressure; and said opening mechanism comprises an electrically conductive wire of high electric resistance, said wire having two ends being adapted to connect to a positive electrode and a negative electrode of a power supply, respectively, and one or more points between said two ends of said wire being attached to the body of said pocket, so that the dilated pocket body will rupture due to melting or weakening of the elastic polymeric material caused by a heat generated at the attached wire, when the ends of said wire are connected to the positive electrode and the negative electrode of the power supply.

7. The device according to claim 3, wherein said pocket is made of an elastic polymeric material, so that the body of said pocket can be dilated with the paste inside said pocket under pressure; and said opening mechanism comprises an electrically conductive wire of high electric resistance, said wire having two ends being adapted to connect to a positive electrode and a negative electrode of a power supply, respectively, and one or more points between said two ends of said wire being attached to the body of said pocket, so that the dilated pocket body will rupture due to melting or weakening of the elastic polymeric material caused by a heat generated at the attached wire, when the ends of said wire are connected to the positive electrode and the negative electrode of the power supply.

8. The device according to claim 1, wherein said pocket is made of an elastic polymeric material, and said opening mechanism comprises a first set of wire holders on an outer surface of said syringe, which are spaced apart along a longitudinal direction of said syringe; a second set of wire holders on said outer surface of said syringe, which are spaced apart along said longitudinal direction of said syringe, wherein an imaginary plane formed by said first set of wire holders and said second set of wire holders divides the syringe into halves; and said thin wire which is slidably received said first set of wire holders and said second set of wire holders with a portion thereof passing across said injection end of said syringe, thereby said pocket can be cut to rupture with a sliding movement of said thin wire.

9. The device according to claim 2, wherein said pocket is made of an elastic polymeric material, and said opening mechanism comprises a first set of wire holders on an outer surface of said syringe, which are spaced apart along a longitudinal direction of said syringe; a second set of wire holders on said outer surface of said syringe, which are spaced apart along said longitudinal direction of said syringe, wherein an imaginary plane formed by said first set of wire holders and said second set of wire holders divides the syringe into halves; and said thin wire which is slidably received said first set of wire holders and said second set of wire holders with a portion thereof passing across said injection end of said syringe, thereby said pocket can be cut to rupture with a sliding movement of said thin wire.

10. The device according to claim 3, wherein said pocket is made of an elastic polymeric material, and said opening mechanism comprises a first set of wire holders on an outer surface of said syringe, which are spaced apart along a longitudinal direction of said syringe; a second set of wire holders on said outer surface of said syringe, which are spaced apart along said longitudinal direction of said syringe, wherein an imaginary plane formed by said first set of wire holders and said second set of wire holders divides the syringe into halves; and said thin wire which is slidably received said first set of wire holders and said second set of wire holders with a portion thereof passing across said injection end of said syringe, thereby said pocket can be cut to rupture with a sliding movement of said thin wire.

11. The device according to claim 1, wherein said pocket is made of an elastic polymeric material, and said opening mechanism comprises a thin tube on an outer surface of said syringe along a longitudinal direction of said syringe; and a knife slidably received in said thin tube, said knife having a retractable blade and a rod connected to said retractable blade at one end thereof, so that said retractable blade received in said tube is able to protrude from said injection end of said syringe by pushing the rod, and thus said pocket can be cut to rupture by said retractable blade, and that said protruding retractable blade can be retracted by pulling the rod.

12. The device according to claim 2, wherein said pocket is made of an elastic polymeric material, and said opening mechanism comprises a thin tube on an outer surface of said syringe along a longitudinal direction of said syringe; and a knife slidably received in said thin tube, said knife having a retractable blade and a rod connected to said retractable blade at one end thereof, so that said retractable blade received in said tube is able to protrude from said injection end of said syringe by pushing the rod, and thus said pocket can be cut to rupture by said retractable blade, and that said protruding retractable blade can be retracted by pulling the rod.

13. The device according to claim 3, wherein said pocket is made of an elastic polymeric material, and said opening mechanism comprises a thin tube on an outer surface of said syringe along a longitudinal direction of said syringe; and a knife slidably received in said thin tube, said knife having a retractable blade and a rod connected to said retractable blade at one end thereof, so that said retractable blade received in said tube is able to protrude from said injection end of said syringe by pushing the rod, and thus said pocket can be cut to rupture by said retractable blade, and that said protruding retractable blade can be retracted by pulling the rod.
